(19) United States
(12) Patent Application Publication
(10) Pub. No.: US 2017/0266035 A1
(43) Pub. Date: Sep. 21, 2017

(54) WIRELESS-CONTROLLED THERAPEUTIC WARMING EYE WEAR DEVICE

(71) Applicant: Brian Young Ming Kuo, Fremont, CA (US)

(72) Inventor: Brian Young Ming Kuo, Fremont, CA (US)

(21) Appl. No.: 15/169,651

(22) Filed: May 31, 2016

Related U.S. Application Data
(63) Continuation-in-part of application No. 15/075,203, filed on Mar. 21, 2016.

(51) Int. Cl. A61F 7/00; A61F 7/02

(52) U.S. Cl. CPC A61F 7/007; A61F 7/02; A61F 2007/0004; A61F 2007/0096; A61F 2007/008; A61F 2007/0094; A61F 2007/0228

(57) ABSTRACT

A therapeutic eye mask having a wireless temperature and duration controller is disclosed. The therapeutic eye mask comprises an eye mask configured to fit over an user's face, a heater and temperature sensor attached to the eye mask configured to heat and sense a temperature, and a wireless thermal controller coupled to the heater and temperature sensor configured to control the heater and temperature sensor to a particular temperature in response to a heat setting on the wireless temperature and duration controller. Such an arrangement allows a user to operate the wireless temperature and duration controller to set the heater and temperature sensor so as to achieve therapeutic warmth and flexible regulation of the temperature and long keeping the temperature constant to the therapeutic eye mask.

### RELATED APPLICATION

This is a continuation-in-part of U.S. Ser. No. 15/075,203 filed Mar. 21, 2016 entitled BLUETOOTH-CONTROLLED ELECTRICAL HEATING PAD DEVICE which is incorporated herein by reference.

### TECHNICAL FIELD OF THE INVENTION

The present invention relates generally to a Bluetooth-controlled electrical heating pad device, and more particularly to a Bluetooth-controlled electrical heating pad device, that rapidly generates heat and has flexible adjustment of device temperature.

### DESCRIPTION OF THE PRIOR ART

Conventional therapeutic warmth-keeping eye wear do not maintain a consistent and constant heat that are often desired with heat therapy. Heat therapy can help relieve migraine headaches that plague many people young and old to relieving day to day stress. Heat therapy is often applied to a person's skin which has many nerve endings. When the heat is applied to the skin, the nerves endings respond to the changes in the skin temperature. Heat has a therapeutic effect on the skin and its nerve endings that tends to relax the surrounding muscles, reduce inflammation, and increase blood flow to the area by widening or dilating blood vessels. Heat also reduces muscle spasms, stimulates blood flow that can change a person's reaction to pain. For example, increased blood flow brings about increased oxygen, proteins, and other nutrients to the area experiencing the pain which has been known to help relieve the pain.

Conventional heating products, such as heat pads and electronic heat pads that are known, although providing an effect of fast heating, do not have a measure of effective control of temperature and may cause undesired burning. The electrical heat pad can be manually shut down to stop heating, yet similar problems still occur often resulting from improper operations.

There is clothing that is available in the market and comprising built-in heating devices; however, in addition to the issue of temperature, it suffers laundry cleaning issues.

For example, Taiwan Utility Model M491460 discloses a temperature-controllable heating device, which has a structure composed of a heating pad, a temperature control mechanism, and a battery. The temperature control mechanism comprises a temperature regulation member, a temperature display member, and a microprocessor. The device uses the temperature control mechanism to control the temperature of the heating pad. Although the temperature display member allows for visual observation of the temperature, stabilization of the temperature may only achieved with a collaborative operation of the temperature regulation mechanism and the microprocessor so that it is not possible to achieve an effect of maintaining constant temperature through automatic detection of clothing temperature and feedback of temperature signal.

Thus, the same issues as those occurring with the electronic heat pad exist in actual applications.

Thus, the above-discussed warm-keeping devices suffers the following shortcomings and drawbacks that require further improvements:

(1) The performance of warm keeping is poor and it is impossible to efficiently increase the body temperature of a user.

(2) It is not possible to provide stable and effective control of temperature and thus accident damages, such as burning, may result.

(3) Effective cleaning and sanitizing are inconvenient for wearables with built-in heating devices.

Thus, it is a challenge for the present inventor and those involved in the business to provide a solution for the above problems.

### SUMMARY OF THE INVENTION

The present invention aims to provide a Bluetooth-controlled warming eye mask that enables fast heat generation and flexible adjustment of clothing temperature.

An object of the present invention is to enable combination of a heating device to an eye mask and effective control a temperature of the heating device combined with the eye mask to thereby achieve efficient temperature rise and long-kept constant temperature.

Another object of the present invention is using an operation device to control a temperature of a heating device in a wireless Bluetooth manner so as to achieve convenience of operation.

To achieve the above objects, the present invention comprises: an eye mask, a Bluetooth control device received in a heating device attached to the eye mask and electrically connected with the Bluetooth control device. The Bluetooth control device and the heating device are electrically connected. A user, when wearing the therapeutic eye mask, may operate a controller to establish, in wireless Bluetooth manner, information connection with the Bluetooth control device and to set a desired temperature and time period for heating. Then, a collaborative cooperation among the Bluetooth control device, the heating device, conducts instantaneous detection of the temperature of the eye mask for automatically maintaining the desired temperature of the eye mask set by the user.

The technique provided above helps overcome the drawbacks of the prior art where the known warm-keeping eye mask has poor warm-keeping performance, is incapable of maintaining a set temperature for long duration, and may cause safety concern due to being incapable of effectively controlling the temperature and may achieve the above-mentioned objects.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is an illustration of an exemplary therapeutic eye mask in accordance to an embodiment of the present invention.

FIG. 2 is a functional block diagram illustrating the therapeutic eye mask in accordance to an embodiment of the present invention.

FIG. 3 is an illustration of an exemplary flow diagram of an operation of the therapeutic eye mask in accordance to an embodiment of the present invention.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

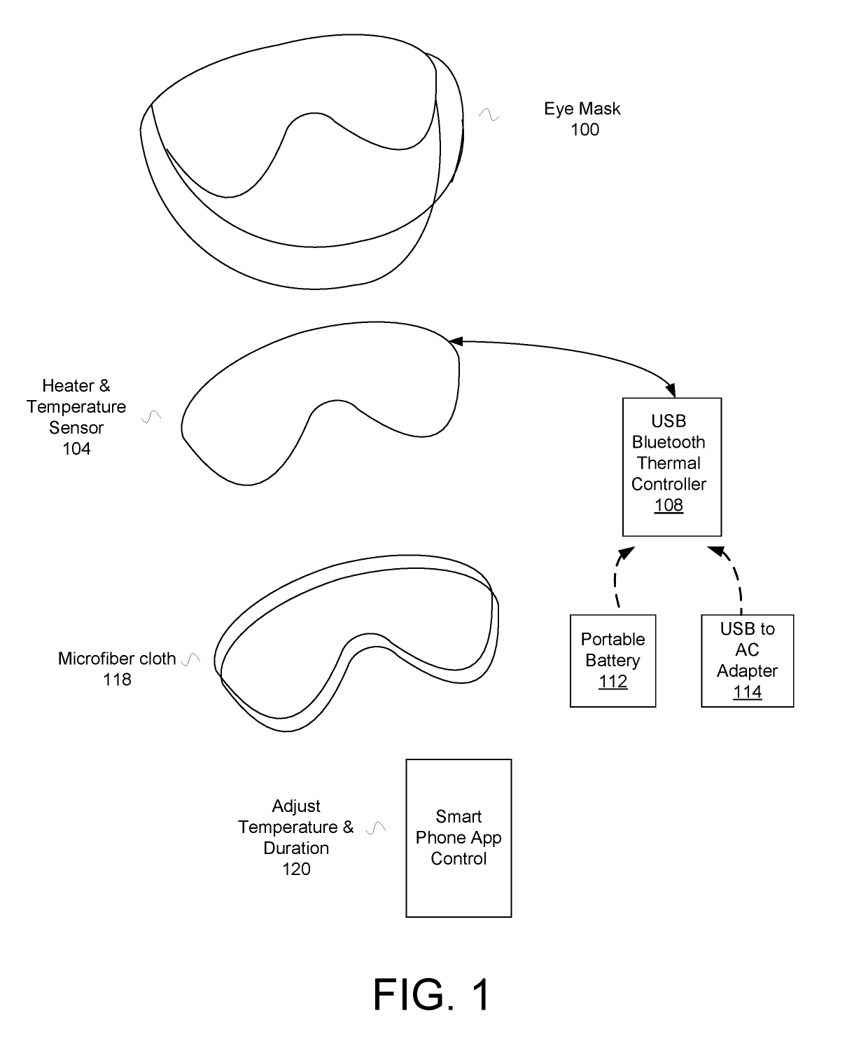

FIG. 1, is an illustration of an exemplary therapeutic eye mask in accordance to an embodiment of the present invention. The therapeutic eye mask includes an eye mask 100, a heater and temperature sensor 104 configured to fit within and about the eye mask, a USB Bluetooth thermal controller 108 being electrically connected to the heater and temperature sensor 104 and to a portable battery 112 electrical power supply or alternatively to an AC adapter 114 electrical power supply to supply electricity to power the USB Bluetooth thermal controller 108 and the heater and temperature sensor 104, a microfiber cloth 118 that provides a moisture source and configured to mate with the heater and temperature sensor 104, and a temperature and duration controller 120. According to an embodiment of the present invention, the temperature and duration controller 120 can be a smart phone running an App to control the eye mask heating temperature and the eye mask heating duration.

The eye mask 100 can include a band which may be elastic to secure and compress the therapeutic eye mask against a user's face. It should be noted that the eye mask 100 and its heater and temperature sensor 104 may be larger to cover more or substantially all of a user's face, or area to be treated. Accordingly, the mask 100 can be a therapeutic heating pad to treat other areas of a user's body.

The heater and temperature sensor 104 can be built into eye mask 100, fit within the perimeter of the eye mask 100, or sandwiched between the eye mask 100 and the user.

In accordance to an embodiment of the present invention, the eye mask 100 and/or the heater and temperature sensor 104 contain thermal absorbed material that can cooled in a freezer and applied for cooling therapeutic effect.

The microfiber cloth 118 can be any medium that is capable of retaining moisture such as a sponge, cotton absorbent material, bath towel. The microfiber cloth 118 can be attached to the heater and temperature sensor 104 using Velcro, a clip, or by sandwiching, overlapping the microfiber to wrap around the eye mask 100 and/or around the heater and temperature sensor 104. The moisture retaining property of the microfiber cloth 118 facilitates greater heat transfer efficiency to the skin of the user compared to an eye mask without a moisture source to facilitate the heat treatment. Accordingly, moistened microfiber cloth 118 along with heat from the heater and temperature sensor 104 affords enhanced relaxing, healing, enjoyable, warmth sensation to the user. Heat moisture treatment applied around the eyes of the user can relieve sore, dry eyes, stress, and migraine headaches. Heat moisture treatment applied to other parts of the user body can relieve aches, pains, enhance blood flow, and therapeutic effects.

In accordance to an embodiment of the present invention, the microfiber cloth 118 includes properties that enable the microfiber cloth 118 to be moisten, cleaned, dried, repeatedly. The microfiber cloth 118 is disposable and replaceable.

In accordance to another embodiment of the present invention, the microfiber cloth 118 can be infused with essential oils such as mint, eucalyptus, lavender, peppermint, or other for aroma therapy in collaboration with the heat therapy to achieve a synergistic enhanced effect from the therapeutic eye mask.

In accordance to an embodiment of the present invention, the heater and temperature sensor 104 includes a USB connector configured to electrically communicate with the USB Bluetooth thermal controller 108. The USB Bluetooth thermal controller 108 includes a second USB connector configured to electrically communicate with the portable battery 112 or AC adapter 114 for an electrical power source.

The portable battery 112 of the present invention can be a regular mobile power supply, which allows a user to supply electrical power to the present invention, when it is not used for recharging a mobile device and thus, no additional power supply device is necessary.

Optionally, the AC adapter 114 converts an AC power source for connection to the heater and temperature sensor 104 using the USB port in accordance to an embodiment of the present invention.

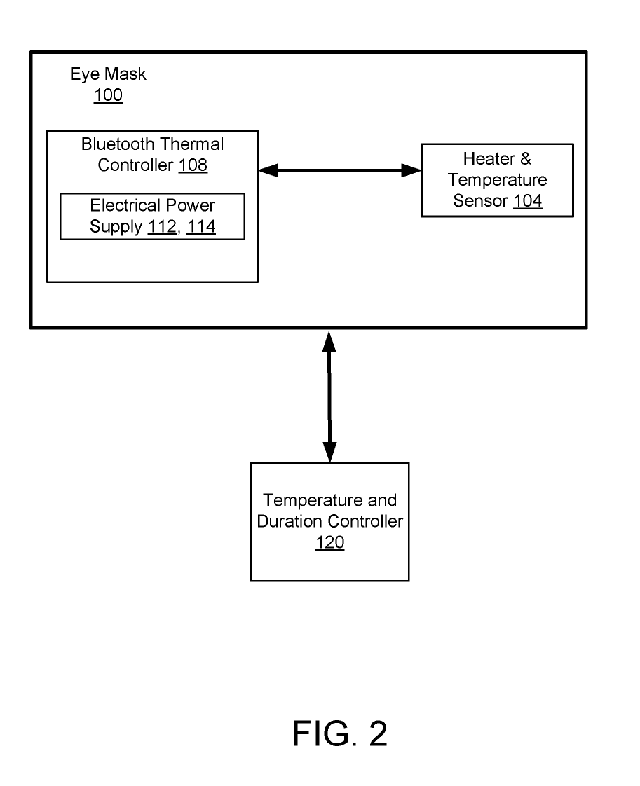

FIG. 2 is a block diagram illustrating the eye mask system in accordance to an embodiment of the present invention. Electrical power supply 112, 114 provides electrical power to the eye mask 100 which receives therein the Bluetooth thermal controller 108, the heater and temperature sensor 104 and with collaborative cooperation among the Bluetooth thermal controller 108, the heater and temperature sensor 104, and input from the temperature and duration controller 120, instantaneous detection of the temperature of the eye mask 100 can be determined and feedback from the heater and temperature sensor 104 can be monitored to provide automatic maintenance of a temperature of the eye mask 100.

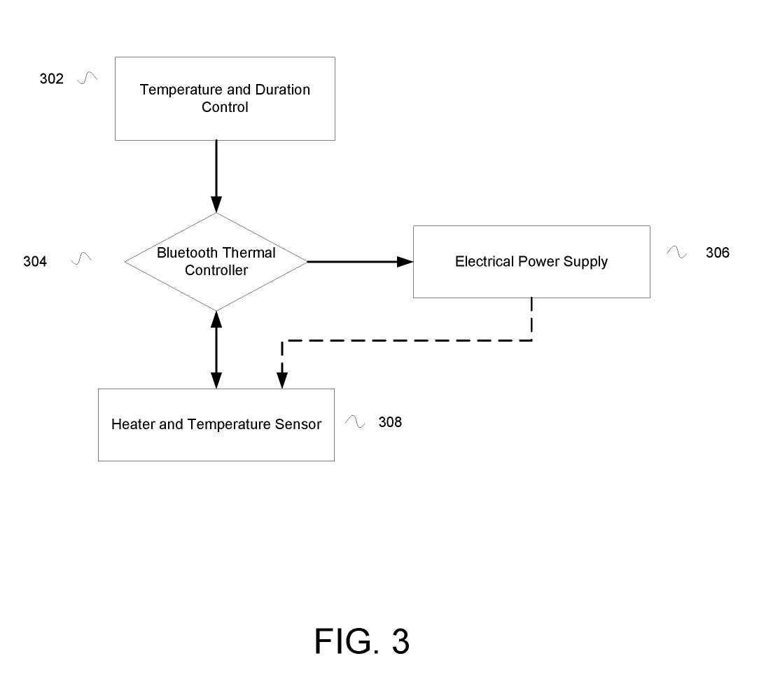

FIG. 3 illustrates an exemplary flow diagram of an operation of the eye mask in accordance to an embodiment of the present invention. The flow diagram begins with step 302. A user operates the temperature and duration controller 120 which in the following example may be a smart phone running an APP program installed that is connected, in a wireless Bluetooth manner, to the Bluetooth thermal controller 108 to allow the user to set a desired temperature as the user wishes and to activate a constant temperature mode. In step 304, the Bluetooth thermal controller 108 issues a temperature signal to cause the heater and temperature sensor 104 to operate and begin heating by connecting the electrical power supply 112, 114 to the heating and temperature sensor 104. Next step 306, in response to the heating and temperature sensor 104, the electrical power supply 112, 114 supplies power to the heater and temperature sensor 104 shown in dashed lines to signify that the electrical power flow can be regulated by the Bluetooth thermal controller 108. In step 308, the heater and temperature sensor 104 receives a connection to the electrical power supply for heat and temperature sensor operation.

Accordingly, during the process of heating, the heater and temperature sensor 104 instantaneously and/or constantly detect the temperature of the eye mask 100. The temperature of the heater is adjusted accordingly based on the feedback from the temperature sensor to the Bluetooth thermal controller 108. For example, when the temperature information is higher than the temperature level set by the user then the heating and temperature sensor 104 is shut down to allow for dropping of temperature; on the other hand, if the temperature information is lower than the level of temperature set by the user, then the heater and temperature sensor 104 is kept on for further and continuous heating. In this way, a long-term effect of keeping the temperature of the eye mask 100 constant may be achieved and operation safety is also ensured.

Referring again to step 302, the temperature and duration controller 120 includes a duration control that can be used to control the Bluetooth thermal controller 108 to turn off the heater and temperature sensor 108 after a preset duration in a timed mode of operation. Accordingly, the therapeutic eye mask can be set to automatically shut off after a predetermined time period.

Although the present invention has been described using Bluetooth for wireless operation, those skilled in the art will appreciate that other wireless connectivity technologies can be similarly adapted to work and is considered within the spirit of the present disclosure. Other wireless connectivity technologies include Near Field Communication (NFC), ANT+. Moreover, the USB connector has been described as the connector in the present invention; however, it is contemplated that those skilled in the art will appreciate that other suitable connectors can be substituted for the USB connector are still within the spirit of the present invention.

The above provides only the description of preferred embodiments of the present invention, which are not for constraining the scope of the present invention. Simple modifications and equivalent variations based on the disclosure made in the specification and the drawings are considered within the scope of the present invention defined in the appended claims

Thus, with reference to all the drawings, the present invention, when put into use, provides the following advantages over the prior art:

(1) A heater and temperature sensor 104 is combined with a therapeutic eye mask; and temperature of the therapeutic eye mask caused by the heater and temperature sensor 104 can be effectively controlled to achieve the purposes of fast temperature rise, long-term maintenance of constant temperature, and operation safety.

(2) A temperature and duration controller 120 is operable to control, in a wireless Bluetooth manner, the temperature of the heater and temperature sensor 104 so as to provide convenience of operation.

(3) The arrangement of the removable microfiber cloth 118 facilitates the use of essential oils for aroma therapy in addition to thermal therapy for relaxation, stress relief, or migraine headache relief.

(4) The arrangement of the removable microfiber cloth 118 can be washed, cleaned, sanitized, or replaced with a new microfiber cloth to maintain a sanitized therapeutic eye mask before each application.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the claims of the present invention.

I claim:

1. A therapeutic eye mask having a wireless temperature and duration controller, comprising:
an eye mask configured to fit over an user's face;
a heater and temperature sensor attached to the eye mask configured to heat and sense a temperature; and
a wireless thermal controller coupled to the heater and temperature sensor configured to control the heater and temperature sensor to a particular temperature in response to a heat setting on the wireless temperature and duration controller.

2. The therapeutic eye mask of claim 1 further comprising a pad medium coupled to the heater and temperature senor configured to retain moisture in the pad medium.

3. The therapeutic eye mask of claim 2, wherein the pad medium is a microfiber cloth.

4. The therapeutic eye mask of claim 1 further comprising a power supply coupled to the wireless thermal controller configured to supply electrical power to the wireless thermal controller and the heater and temperature sensor.

5. The therapeutic eye mask of claim 4, wherein the power supply is a portable battery.

6. The therapeutic eye mask of claim 4, wherein the power supply is an AC source.

7. The therapeutic eye mask of claim 4 further comprising a plurality of USB connectors configured to couple the heater and temperature sensor with the wireless thermal controller and the power supply.

8. The therapeutic eye mask of claim 1, wherein the wireless temperature and duration controller and the wireless thermal controller communicate using Bluetooth technology.

9. The therapeutic eye mask of claim 1, wherein the wireless temperature and duration controller includes an App configured to operate from a smart phone.

10. The therapeutic eye mask of claim 1, wherein the wireless temperature and duration controller is configured to set a selectable heat setting for the heater and temperature sensor.

11. The therapeutic eye mask of claim 1, wherein the wireless temperature and duration controller is configured to set a selectable time duration to operate the heater and temperature sensor.

12. The therapeutic eye mask of claim 1, wherein the eye mask includes an elastic band configured to compress the eye mask against the user's face.
